(19) United States
(12) Patent Application Publication
(10) Pub. No.: US 2025/0049450 A1
(43) Pub. Date: Feb. 13, 2025

(54) ORTHOPEDIC INSTRUMENTS AND METHODS

(71) Applicant: Paragon 28, Inc., Englewood, CO (US)

(72) Inventors: Joseph DOGUÉ, Aurora, CO (US); Michael SCHMIDT, Greenwood Village, CO (US); Laura Zagrocki BRINKER, Lone Tree, CO (US); Mansour Adel ALBAYYAT, Tucson, AZ (US); Brandon Michael MARTIN, Tucson, AZ (US); Makenna AITKEN, Tucson, AZ (US); Deip Ashok SEKHADIA, Tucson, AZ (US); Tai James COMBS, Queen Creek, AZ (US)

(73) Assignee: Paragon 28, Inc., Englewood, CO (US)

(21) Appl. No.: 18/931,929

(22) Filed: Oct. 30, 2024

Related U.S. Application Data
(63) Continuation of application No. PCT/US2023/066491, filed on May 2, 2023.
(60) Provisional application No. 63/363,963, filed on May 2, 2022.

(51) Int. Cl. A61B 17/17; A61B 17/16

(52) U.S. Cl. CPC A61B 17/1775; A61B 2017/1602

(57) ABSTRACT

An orthopedic instrument system that includes a burr and a burr guide. The burr guide includes an elongated opening, in which at least a portion of the elongated opening includes a substantially circular opening having a greater dimension than that of the adjacent portion of the elongated opening. The circular opening is configured to receive at least a portion of the burr therein and therethrough such that the burr may be manipulated along the length of the elongated opening. The burr has a shaft and a first end opposite a second end with the first end being configured to facilitate being releasably coupled to a tool. The second end of the burr has an engagement feature that is configured to cut and/or resurface a bone surface. A method of using the orthopedic instrument system is also disclosed herein.

### CROSS REFERENCE TO RELATED APPLICATION

This application is a bypass continuation of PCT Application No. PCT/US2023/066491, filed May 2, 2023, and entitled “Orthopedic Instruments and Methods,” which claims priority benefit under 35 U.S.C. § 119 (e) to U.S. Provisional Patent Application No. 63/363,963, filed on May 2, 2022, and entitled “Orthopedic Instruments and Methods” the disclosure of these applications are hereby incorporated herein by reference in their entirety.

### TECHNICAL FIELD

The present disclosure relates to instruments, systems, and methods associated with performing orthopedic procedures. The present disclosure relates to podiatric and orthopedic instruments, systems, and methods related to procedures including but not limited to arthroplasty, arthrodesis, arthroeresis and/or osteotomies of joints in the foot/ankle and/or procedures incorporating surrounding bones/soft tissue.

### BACKGROUND OF THE INVENTION

Many currently available instruments, systems, devices, and methods for addressing bone, soft tissue, and joint trauma (acute and chronic, e.g., defect, gradual deterioration, deformity, etc.) do not completely address the needs of patients. Additionally, many currently available instruments, systems, devices, and methods for addressing joint trauma and/or other conditions fail to account for properties of joint anatomy and associated mechanical and kinematic movement patterns/capabilities.

### SUMMARY OF THE INVENTION

The present disclosure is directed towards an orthopedic instrument system, according to one aspect of the present disclosure. The system includes a burr and a burr guide. The burr guide includes an elongated opening, wherein at least a portion of the elongated opening includes a substantially circular opening having a greater lateral dimension than that of the adjacent portion of the elongated opening. The circular opening is configured to receive at least a portion of the burr therein and therethrough such that the burr may be manipulated along the length of the elongated opening.

According to one aspect of the present disclosure, the guide includes a body from which at least one lobe extends laterally.

According to one aspect of the present disclosure, the at least one lobe includes a pair of lobes extending laterally from opposite sides of the body.

According to one aspect of the present disclosure, each of the pair of lobes includes a slot positioned in a central portion of the respective lobes, wherein the slots extend from a top surface through to a bottom surface of the respective lobes.

According to one aspect of the present disclosure, the system includes at least one stabilization wire configured to releasably couple the guide with a bone of a patient.

According to one aspect of the present disclosure, the guide is substantially symmetrical about at least one plane extending therethrough.

According to one aspect of the present disclosure, the at least one plane intersects or is coplanar with a longitudinal axis extending along a length of the elongated opening.

According to one aspect of the present disclosure, the burr includes a first end opposite a shaft from a second end, wherein the first end comprises a geometry configured to releasably couple with a powered instrument.

According to one aspect of the present disclosure, the second end of the burr includes an engagement feature.

According to one aspect of the present disclosure, the engagement feature includes a geometry that is greater in at least one dimension than that of the shaft and the first end of the burr.

The present disclosure is directed toward an instrument. The instrument includes an upper portion, a central portion having a greater lateral dimension than that of the upper portion, and a lower portion having a greater lateral dimension than the central portion.

According to another aspect of the present disclosure, the second end includes an engagement feature, wherein the engagement feature includes a geometry that is greater in at least one lateral dimension than that of the shaft or the first end.

According to another aspect of the present disclosure, the engagement feature is configured to function as a mechanical stop, and further includes a substantially cylindrical geometry having a plurality of members protruding from an outer surface of the engagement feature which extends for a length and/or circumference of the outer surface, wherein the plurality of members may extend in an outward direction from the outer surface at an angle that is at least one of orthogonal or oblique relative to the outer surface.

The present disclosure is directed toward a guide. The guide includes a body which includes an elongated opening extending from a top surface through to a bottom surface of the body, and a circular opening extending from a top surface through to a bottom surface of the body. The guide also includes a pair of lobes extending laterally from the body.

According to another aspect of the present disclosure, the elongated opening and the circular opening are in fluid communication with one another.

According to another aspect of the present disclosure, the circular opening comprises a lateral dimension that is greater than a greatest lateral dimension of the elongated slot.

According to another aspect of the present disclosure, each of the lobes includes a slot extending from a top surface through to a bottom surface of the respective slot.

According to another aspect of the present disclosure, each of the lobes includes a substantially curved geometry.

According to another aspect of the present disclosure, the guide is symmetrical about a plane that overlaps with a longitudinal axis extending along a length of the elongated slot.

According to another aspect of the present disclosure, the elongated slot includes a depression extending along at least a portion of a length of the elongated slot, wherein the depression is positioned adjacent the top surface of the body.

### BRIEF DESCRIPTION OF THE DRAWINGS

The accompanying drawings, which are incorporated in and constitute a part of the specification, illustrate embodiments of the inventions and together with the detailed description herein, serve to explain the principles of the inventions. It is emphasized that, in accordance with the standard practice in the industry, various features may or may not be drawn to scale. In fact, the dimensions of the various features may be arbitrarily increased or reduced for clarity of discussion. The drawings are only for purposes of illustrating embodiments of inventions of the disclosure and are not to be construed as limiting the inventions.

FIG. 1 is a front perspective view of an orthopedic instrument system, in accordance with the present disclosure;

FIG. 2 is a front view of the orthopedic instrument system of FIG. 1, in accordance with the present disclosure;

FIG. 3 is an alternate front perspective view of the orthopedic instrument system of FIG. 1, in accordance with the present disclosure;

FIG. 4 is a rear perspective view of the orthopedic instrument system of FIG. 1, in accordance with the present disclosure;

FIG. 5 is an alternate rear perspective view of the orthopedic instrument system of FIG. 1, in accordance with the present disclosure;

FIG. 6 is a front perspective view of the orthopedic instrument system of FIG. 1 in a second position, in accordance the present disclosure;

FIG. 7 is a first side perspective view of the orthopedic instrument system of FIG. 6, in accordance with the present disclosure;

FIG. 8 is a second side perspective view of the orthopedic instrument system of FIG. 6, in accordance with the present disclosure;

FIG. 9 is a front, top view of an orthopedic instrument of the orthopedic instrument system of FIGS. 1 and 6, in accordance with the present disclosure;

FIG. 10 is a front, bottom view of the orthopedic instrument of FIG. 9 of the orthopedic instrument system of FIGS. 1 and 6, in accordance with the present disclosure;

FIG. 11 is a rear, top view of the orthopedic instrument of FIG. 9 of the orthopedic instrument system of FIGS. 1 and 6, in accordance with the present disclosure;

FIG. 12 is a rear, bottom view of the orthopedic instrument of FIG. 9 of the orthopedic instrument system of FIGS. 1-6, in accordance with the present disclosure;

FIG. 13 is a first side view of the orthopedic instrument of FIG. 9 of the orthopedic instrument system of FIGS. 1-6, in accordance with the present disclosure; and

FIG. 14 is a second side view of the orthopedic instrument of FIG. 9 of the orthopedic instrument system of FIGS. 1 and 6, in accordance with the present disclosure.

### DETAILED DESCRIPTION FOR CARRYING OUT THE INVENTION

In this detailed description and the following claims, the words proximal, distal, anterior, or plantar, posterior, or dorsal, medial, lateral, superior, and inferior are defined by their standard usage for indicating a particular part or portion of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regards to the foot, the term “dorsal” refers to the top of the foot and the term “plantar” refers the bottom of the foot.

Similarly, positions or directions may be used herein with reference to anatomical structures or surfaces. For example, as the current implants, devices, instrumentation, and methods are described herein with reference to use with the bones of the foot, the bones of the foot, ankle and lower leg may be used to describe the surfaces, positions, directions or orientations of the implants, devices, instrumentation, and methods. Further, the implants, devices, instrumentation, and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, instrumentation, and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, instrumentation, and methods, and the aspects, components, features and the like thereof, described herein with respect to the right foot may be mirrored so that they likewise function with the left foot. Further, the implants, devices, instrumentation, and methods, and the aspects, components, features and the like thereof, disclosed herein are described with respect to the foot for brevity purposes, but it should be understood that the implants, devices, instrumentation, and methods may be used with other bones of the body having similar structures.

The instruments, implants, systems, assemblies, and related methods for maintaining, correcting, and/or resurfacing joint surfaces of the present disclosure may be similar to, such as include at least one feature or aspect of, the implants, systems, assemblies and related methods disclosed in U.S. Pat. No. 10,117,749, issued on Nov. 6, 2018 and entitled “Subtalar Joint Implant”; European Patent No. 3756626 issued on Dec. 30, 2020 and entitled “Subtalar Joint Implant”; European Patent Application No. 15770960.1A filed on Jul. 15, 2020 and entitled “Subtalar Joint Implant”; U.S. patent application Ser. No. 17/653,029, filed on Mar. 1, 2022 and entitled “Methods for Performing an Arthroplasty of the Subtalar Joint”; PCT Application No. PCT/US2022/071638 filed Mar. 30, 2022 and entitled “Orthopedic Implants and Methods”; U.S. patent application Ser. No. 17/657,522 filed on Mar. 31, 2022 and entitled “Systems and Methods for Controlled Facet Repositioning in the Calcaneus”; U.S. Provisional Patent Application No. 63/362,850 filed on Apr. 12, 2022 and entitled “Surgical Methods for Procedures of the Subtalar Joint”; and U.S. Provisional Patent Application No. 63/362,853 filed on Apr. 12, 2022 and entitled “Orthopedic Implants and Instruments”; which are all hereby incorporated herein by reference in their entireties.

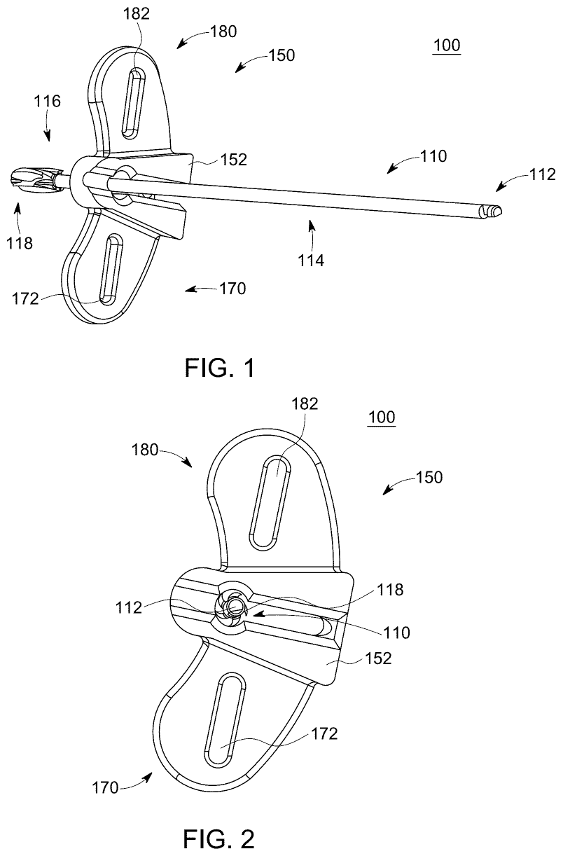

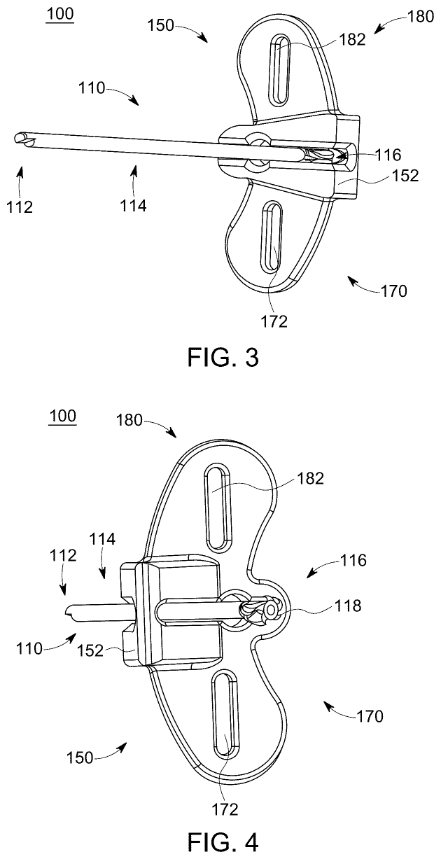

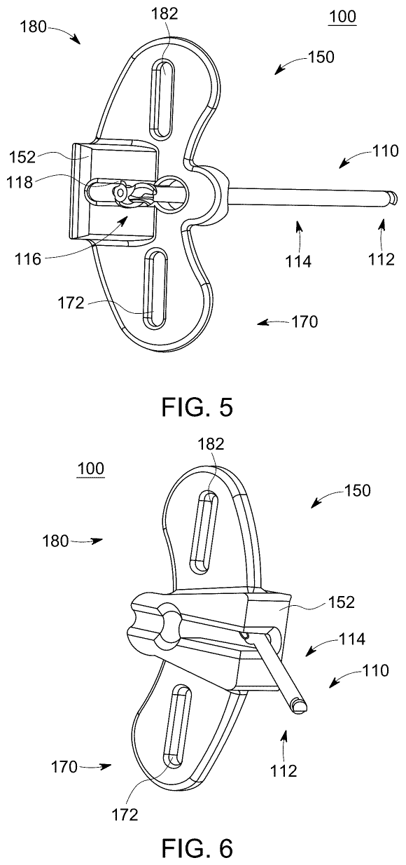

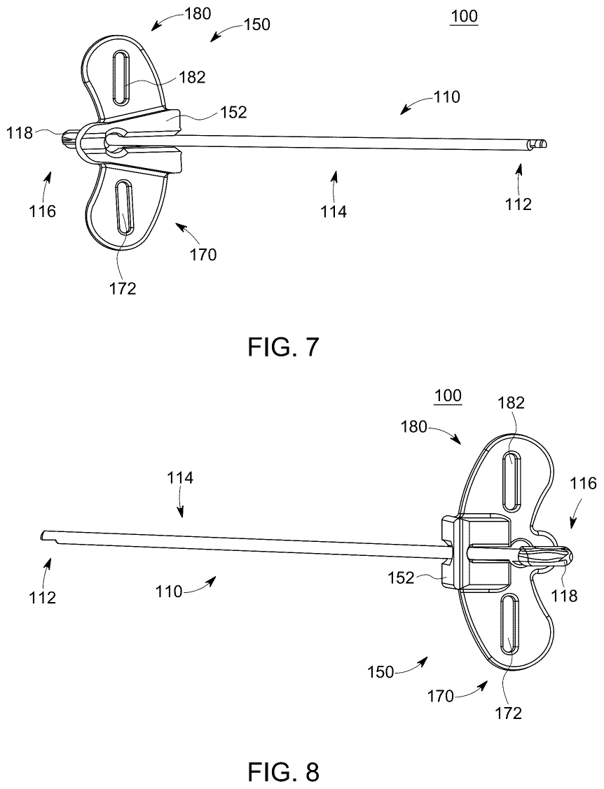

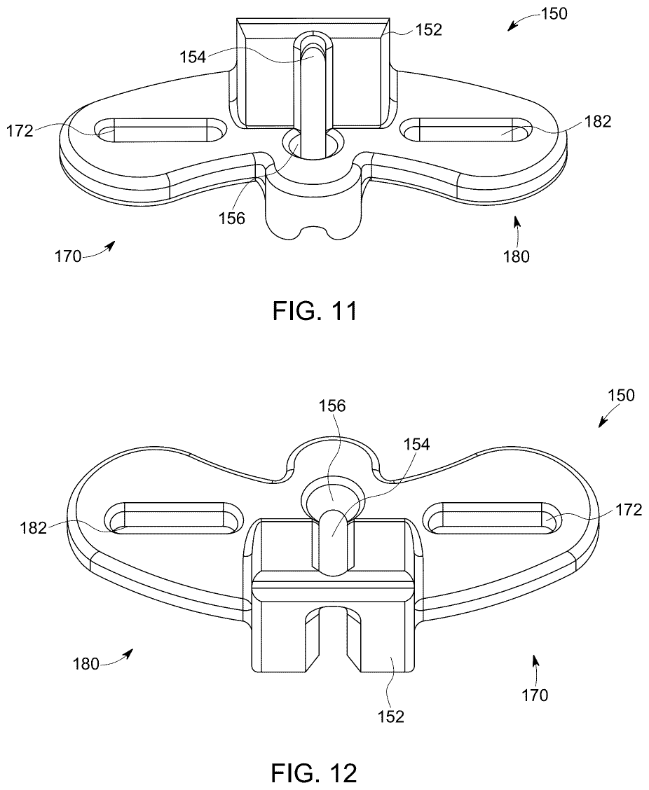

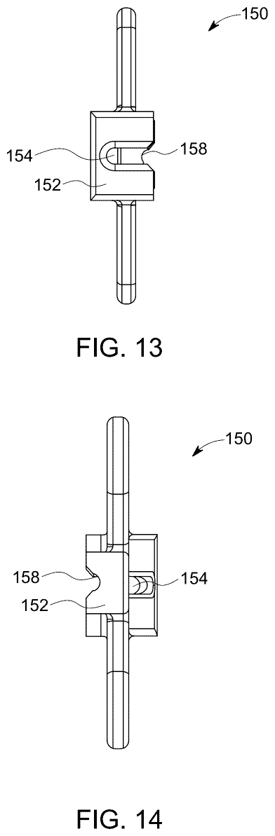

Referring to the drawings, wherein like reference numerals are used to indicate like or analogous components throughout the several views, and with particular reference to FIGS. 1-14, an orthopedic instrumentation system 100 (referred to hereinafter as “system 100” for the sake of brevity) is shown, according to one aspect of the present disclosure. The system 100 may be implemented in conjunction with one or more additional instrument systems, implant systems, and/or other surgical systems common to orthopedic procedures. For example, the system 100 may be implemented in conjunction with a robotic surgery system, various resection guides/systems, alignment guides/systems, and/or implants/implant systems. Further, in some aspects additional components may be included with the system 100 that are not shown and described herein. For example, the system 100 may include various components common to orthopedic procedures such as stabilization wires (e.g., k-wires, olive wires, etc.), pins, fasteners, and other components. In some aspects, one or more components of the system 100 may be manufactured or otherwise produced (e.g., 3-D printed, additively manufactured, etc.) such that the one or more components are specific to an anatomy of a particular patient. In some aspects, imaging data (e.g., CT data, etc.) may be collected and, from the imaging data, one or more components of the system 100 produced such that the components have a geometry that corresponds to at least a portion of an anatomy of a patient (e.g., a complimentary geometry, etc.).

As shown and for example, the system is configured to be positioned adjacent a subtalar joint of a patient from a lateral approach (e.g., adjacent the sinus tarsi of the patient). In some aspects, the system may be configured to releasably couple with one or more components of the anatomy of the patient (e.g., the talus and calcaneus, which are components of/adjacent to the subtalar joint) such that the system does not abut nor interfere with a fibula of a patient.

Referring to FIGS. 1-8, the system 100 is shown to include an instrument 110 and a guide 150, according to one aspect of the present disclosure. The instrument 110 is shown in FIGS. 1-8 as an elongated burr, although in some aspects various alternative instruments may be implemented in conjunction with the guide 150 as part of the system 100. For example, a rasp, various resurfacing tools, and/or various cutting tools may be implemented in conjunction with the guide 150 in a manner that is the same as and/or similar to that shown and described with reference to FIGS. 1-8. Similarly, the instrument 110 may include a burr device that is different than that shown in FIGS. 1-8, for example, a burr with one or more components thereof having different sizes, geometries, additional components, or without components shown and described herein.

The instrument 110 is shown to include a shaft 114 having a first end 112 that is opposite from a second end 116. The first end 112 may include one or more geometries common to releasable coupling systems implemented in orthopedic and other medical specialties surgical instruments, to facilitate releasable coupling with a drill, driver, or other power tool. As shown, the coupling geometry of the first end 112 does not extend a significant length down the shaft 114 toward the second end 116. However, in alternate embodiments the coupling geometry of the first end 112 may extend along at least a portion of the shaft 114 toward the second end 116. The first end 112 is shown to include a lesser lateral dimension than that of the second end 116 and, further, includes a lateral dimension substantially similar and/or equal to that of at least a portion of the shaft 114. As shown, the shaft 114 and the first end 112 include an at least partially cylindrical geometry with an at least partially circular cross-section. However, in alternate embodiments the shaft 114 and the first end 112 may include alternate geometries, for example a rectangular prism with a rectangular cross-section, a hexagonal prism with a hexagonal cross-section, etc.

The second end 116 is shown to include an engagement feature 118 disposed substantially opposite the shaft 114 from the first end 112. As shown, the engagement feature 118 includes a geometry that is greater in at least one lateral dimension (e.g., greater cross-sectional dimension) than that of the shaft 114 or the first end 112. Accordingly, the instrument 110 is configured such that it may be inserted through an opening with a lateral dimension greater than that of the first end 112 and at least a portion of the shaft 114, but lesser than that of the engagement feature 118 of the second end 116. The engagement feature 118 may be configured to function as a mechanical stop, thus abutting a component adjacent the opening with the lateral dimension lesser than that of the engagement feature 118. Accordingly, the instrument 110 and components thereof may be configured to prevent accidental removal of the instrument 110 in its entirety from such an opening.

The engagement feature 118 may include a substantially cylindrical geometry such as that shown in FIGS. 1-8, with various components protruding from the outer surface of the engagement feature 118 along the length and circumference of the outer surface. The engagement feature 118 may include, for example, one or more protrusions extending outward from the outer surface at an angle that is either orthogonal or oblique relative to the outer surface. As shown for example, the engagement feature 118 includes 5 protrusions spaced substantially equidistantly from one another about the circumference of the engagement feature 118 and extending along the length thereof. In some aspects, the engagement feature 118 may include greater or fewer protrusions variously spaced about both the circumference and length of the outer surface of the engagement feature 118 and, furthermore, having various geometries.

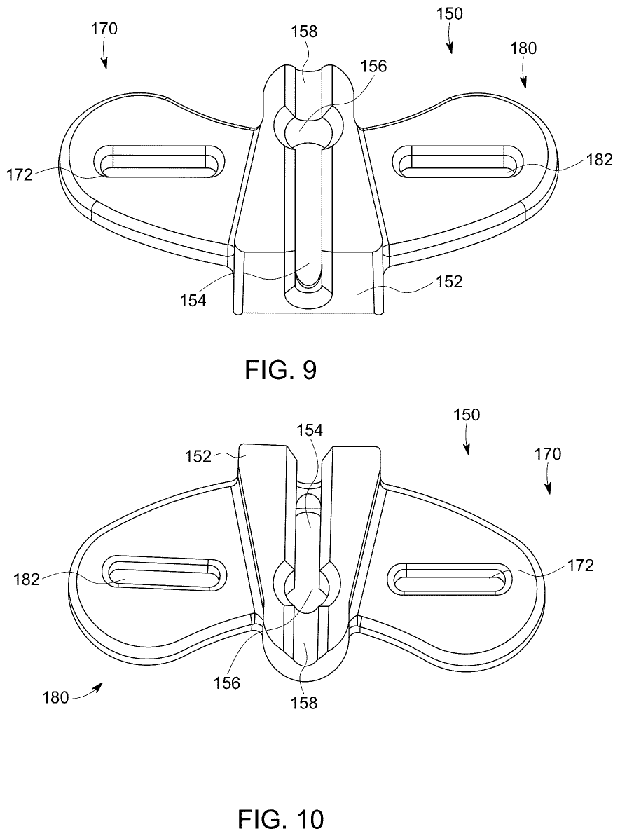

The system 100 is also shown to include the guide 150, shown in FIGS. 1-8 in conjunction with the instrument 110 and independent from the instrument 110 in FIGS. 9-14. The guide 150 may be implemented in conjunction with the instrument 110 or, in some aspects, may be implemented with one or more other components (including but not limited to those common to orthopedic surgical procedures). In some aspects, the guide 150 may be provided separately from the instrument 110 as part of an implant system/kit, an instrument system/kit, a series of instruments, or otherwise as part of a collection of surgical/orthopedic instruments/components. Further, the guide 150 may be provided with additional components not shown and/or described in detail herein, for example stabilization wires or other fixation components configured to releasably couple the guide 150 with one or more bones (or other portions of the anatomy of a patient) or other instruments/components.

The guide 150 is shown to include a body 152 disposed substantially centrally relative (and/or adjacent) to other components of the guide 150, according to one aspect of the present disclosure. As shown, the guide 150 has a substantially curved or C-shaped geometry, with the body 152 positioned centrally. However, in some aspects the guide 150 may have alternate geometries to accommodate a patient's anatomy. The body 152 is shown to include a tapered geometry such that the taper extends from beyond the limit of the convexity of the C-shape (widest portion of the body 152) to within the convexity of the C-shape (narrowest portion of the body 152, which may include a rounded end portion). The body 152 is shown to include an opening 154 shown herein to have a substantially elongated geometry and be disposed in a central portion of the body 152, where the opening 154 extends from a top surface through to a bottom surface of the body 152 (opposite the body 152 from the top surface) so as to provide fluid communication therebetween. Further, the opening 154 is shown to extend laterally across a majority of the lateral dimension of the body 152 (e.g., more than half of the width).

The opening 154 is shown to include a bore 156 disposed adjacent the opening 154 and extending from the top surface through to the bottom surface of the body 152 so as to establish fluid communication therebetween (e.g., similar to that of the opening 154). As shown, the bore 156 is in fluid communication with the opening 154 (e.g., to form a keyhole opening collectively). As shown, the bore 156 includes a substantially cylindrical geometry but in some aspects may include alternate geometries and/or positions. For example, as shown in FIGS. 1-8, at least a portion of the shaft 114 of the instrument 110 may be manipulated from the bore 156 to the opening 154 and vice-versa without the removal of the shaft 114 from the guide 150. In some aspects, the opening 154 may have a lateral dimension greater than that of the shaft 114 and lesser than that of the engagement feature 118 so as to prevent pullout of the engagement feature 118 (and accordingly, the instrument 110) through the opening 154. In some aspects, the bore 156 may have a similar lateral or radial dimension configured to prevent pullout of the engagement feature 118 of the instrument 110 (e.g., as a mechanical stop). However, in some aspects, the bore 156 may include a lateral dimension greater than that of the engagement feature 118 such that the instrument 110 may be inserted (and subsequently removed), engagement feature 118 first (or last in removal), into (or from) the guide 150 via the bore 156 such that a portion of the shaft 114 is disposed within the bore 156. The instrument 110 may then be manipulated throughout a defined range of motion while at least a portion of the shaft 114 is disposed within the bore 156. However, the instrument 110 may also be manipulated laterally such that the shaft 114 (at least a portion thereof) is repositioned from within the bore 156 to within the opening 154. Accordingly, the elongated geometry of the opening 154 allows manipulation of the instrument 110 throughout a different and greater range of motion than when the shaft 114 of the instrument 110 remained within the bore 156.

The body 152 is further shown to include a depression 158 extending along at least a portion of the top surface of the body 152, according to one aspect of the present disclosure. As shown in FIGS. 9-12, the depression 158 includes a footprint on the body 152 (e.g., the top surface thereof) that overlaps with at least a portion of the footprint of the opening 154 and the bore 156. In some aspects, the opening 154 and the depression 158 may have a common longitudinal axis in the lateral direction (e.g., in a plane parallel to the top surface of the body 152. As shown, the body 152 (and the guide 150) are substantially symmetrical about the longitudinal axis of the depression 158. The depression 158 may be sized so as to receive a linear, cylindrical surgical component or fastener (e.g., a k-wire) at least partially therein such that the component may provide an indication as to location and/or alignment of the guide 150 relative to the anatomy of a patient prior to releasably coupling the guide 150 with the anatomy of the patient. Further, when a k-wire or other component is laid within the depression 158, the k-wire indicates a plane along with the instrument 110 may be manipulated when positioned within the opening 154 and/or bore 156 of the guide 150. The guide 150 and the k-wire may then be positioned/repositioned (and checked with fluoroscopy or other imaging techniques) until a verification is made that the plane indicated by the k-wire corresponds with a desired plane along which the instrument 110 will be manipulated. In some aspects for example, this plane may include at least a portion of the posterior face of the calcaneus/talus and/or various other components/tissues of the subtalar joint.

The guide 150 is further shown to include a first lobe 170 and a second lobe 180, where the first and second lobes 170, 180 extend from opposite sides of the body 152 and include a curvature in substantially the same direction so as to form the aforementioned C-shape of the guide 150. As shown, the first and second lobes 170, 180 are of the same/similar geometry and size, although in some aspects it is contemplated that one of the lobes may have a different size and/or geometry than the other. Each of the first and second lobes 170, 180 are shown to have a curvature biased in the direction of the narrower of the two ends of the body 152 (e.g., the end to which the bore 156 is closest) and include a substantially rounded terminal end opposite the point at which the lobes extend from the sides of the body 152. The first and second lobes 170, 180 are shown to include a first slot 172 positioned on the first lobe 170 and a second slot 182 positioned on the second lobe 180. As shown, the first and second slots 172, 182 are centrally positioned relative to the first and second lobes 170, 180, and furthermore are of a substantially similar and/or equal length, width, and overall elongated geometry. The first and second slots 172, 182 may be configured to receive an element therethrough, for example a k-wire or other stabilization component, or a pin or other distraction-compatible linear, rigid body such that once received within the first and second slots 172, 182, the wire, pin, or other rigid body may be translated along the length of the respective slot 172, 182.

In some aspects, the system 100 may be implemented according to one or more surgical methodologies either independently or as a sub-method of a larger surgical procedure. The following methodology is intended to be exemplary and in no way limiting of the applications of the system 100 and/or any components thereof.

In some aspects a physician may select the system 100 (and/or one or more components thereof) for use in a surgical procedure involving the subtalar joint and/or adjacent soft tissue structures. Prior to implementing the system 100 in a procedure (e.g., prior to make incisions, etc.), the physician may position the guide 150 with a k-wire at least partially disposed within the depression 158 on the skin of a patient on the lateral portion of the foot adjacent the subtalar joint. The physician may then implement fluoroscopy or other imaging techniques to determine if the plane indicated by the k-wire aligns with a plane along which the physician wishes to manipulate the instrument 110 (which in this example will be considered a burr as shown and described subsequently herein but may also include additional/alternate cutting or positioning instrumentation). Upon confirmation of the position of the guide 150 and the corresponding plane indicated by the k-wire, the physician may then make one or more incisions and/or percutaneous stab incisions to the lateral portion of the foot adjacent the sinus tarsi. The physician may then place one or more distraction pins (e.g., one in the talus and one in the calcaneus) through the slots 172, 182 of the guide 150 such that each pin is at least partially disposed within one of the slots (with the pins ideally at the portion of the slots 172, 182 closest to the body 152). The physician may then couple a distractor with the pins and, accordingly, distract the talus and calcaneus thereby providing access to the joint space of the subtalar joint. The instrument 110 may then position the engagement feature 118 first through the bore 156 (and into the joint space) and be manipulated through a range of motion defined by the bounds of the bore 156 and the opening 154 such that the engagement feature 118 may be swept at various depths along the plane indicated previously by the k-wire. The instrument 110 may then be coupled at the first end 112 with a driver or other common orthopedic power tool. As the engagement feature 118 is swept through the joint space, the protrusions of the engagement feature are configured to resurface, remove, and resect soft tissue of the joint space and/or portions of the superior portion of the calcaneus (e.g., the posterior facet) and/or the inferior portion of the talus. This process may be performed iteratively with periodic evaluation of the resection/burring performed by the instrument 110. In some aspects, imaging techniques may be implemented in this evaluation. Once a desired amount of “burring” (e.g., resection, resurfacing, soft tissue removal, etc.) has been performed, the instrument 110 may be decoupled from the tool and removed from the guide 150 via the bore 152. The distractor may then be removed from the distraction pins, and subsequently the guide 150 and distraction pins may be removed from the patient.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

What is claimed:

1. An orthopedic instrument system, comprising:
a burr; and
a guide, comprising:
an elongated opening, wherein at least a portion of the elongated opening comprises a substantially circular opening having a greater lateral dimension than that of the adjacent portion of the elongated opening,
wherein the circular opening is configured to receive at least a portion of the burr therein such that the burr may be manipulated along the length of the elongated opening.

2. The orthopedic instrument system of claim 1, wherein the guide comprises a body from which at least one lobe extends laterally.

3. The orthopedic instrument system of claim 2, wherein the at least one lobe comprises a pair of lobes extending laterally from opposite sides of the body.

4. The orthopedic instrument system of claim 3, wherein each of the pair of lobes comprises a slot positioned in a central portion of the respective lobes, wherein the slot extends from a top surface through to a bottom surface of the respective lobes.

5. The orthopedic instrument system of claim 1, further comprising at least one stabilization wire configured to releasably couple the guide to a bone of a patient.

6. The orthopedic instrument system of claim 1, wherein the guide is substantially symmetrical about at least one plane extending therethrough.

7. The orthopedic instrument system of claim 6, wherein the at least one plane intersects or is coplanar with a longitudinal axis extending along a length of the elongated opening.

8. The orthopedic instrument system of claim 1, wherein the burr comprises a shaft having a first end opposite from a second end, wherein the first end comprises a geometry configured to releasably couple with a powered instrument.

9. The orthopedic instrument system of claim 8, wherein the second end of the burr comprises an engagement feature.

10. The orthopedic instrument system of claim 9, wherein the engagement feature comprises a geometry that is greater in at least one dimension than that of the shaft and the first end of the burr.

11. A burr instrument, comprising:
a first end opposite a shaft and a second end, wherein the first end is configured to comprise at least one geometry that facilitates being releasably coupled to a tool.

12. The burr instrument of claim 11, wherein the second end comprises an engagement feature, wherein the engagement feature comprises a geometry that is greater in at least one lateral dimension than that of the shaft or the first end.

13. The burr instrument of claim 12, wherein the engagement feature is configured to function as a mechanical stop, and further comprises a substantially cylindrical geometry having a plurality of members protruding from an outer surface of the engagement feature which extends for a length and/or circumference of the outer surface, wherein the plurality of members extend in an outward direction from the outer surface at an angle that is at least one of orthogonal or oblique relative to the outer surface.

14. A guide, comprising:
a body, comprising:
an elongated opening extending from a top surface through to a bottom surface of the body; and
a circular opening extending from a top surface through to a bottom surface of the body; and
a pair of lobes extending laterally from the body.

15. The guide of claim 14, wherein the elongated opening and the circular opening are in fluid communication with one another.

16. The guide of claim 15, wherein the circular opening comprises a lateral dimension that is greater than a lateral dimension of the elongated slot.

17. The guide of claim 14, wherein each of the lobes comprises a slot extending from a top surface through to a bottom surface of the respective slot.

18. The guide of claim 17, wherein each of the lobes comprises a substantially curved geometry.

19. The guide of claim 14, wherein the burr guide is symmetrical about a plane that overlaps with a longitudinal axis extending along a length of the elongated slot.

20. The guide of claim 14, wherein the elongated slot comprises a depression extending along at least a portion of a length of the elongated slot, wherein the depression is positioned adjacent the top surface of the body.
